(19) United States
(12) Patent Application Publication
(10) Pub. No.: US 2025/0345375 A1
(43) Pub. Date: Nov. 13, 2025

(54) LACTOBACILLUS CRISPATUS COMPOSITION FOR USE IN PREGNANT SUBJECTS

(71) Applicants: Imperial College Innovations Limited, London (GB); Osel, Inc., Mountain View, CA (US)

(72) Inventors: Phillip R. BENNETT, London (GB); David A. MACINTYRE, London (GB); Thomas P. PARKS, Mountain View, CA (US)

(21) Appl. No.: 18/277,762

(22) PCT Filed: Feb. 18, 2022
(86) PCT No.: PCT/GB2022/050453

§ 371 Date: Aug. 17, 2023

Related U.S. Application Data
(60) Provisional application No. 63/151,474, filed on Feb. 19, 2021.

(30) Foreign Application Priority Data
Mar. 8, 2021 (GB) 2103170.3

(51) Int. Cl. A61K 35/747; A61P 15/06

(52) U.S. Cl. CPC A61K 35/747; A61P 15/06

(57) ABSTRACT

The present invention relates to a composition for use in pregnant subjects comprising Lactobacillus crispatus cells, wherein said composition minimises and/or prevents pregnancy complications, such as miscarriage, preterm labour and preterm birth.

### FIELD OF INVENTION

The present invention relates to a composition for use in pregnant subjects, wherein said composition minimises and/or prevents pregnancy complications, such as miscarriage, preterm labour and preterm birth.

### SEQUENCE LISTING

A Sequence Listing conforming to the rules of WIPO Standard ST.25 is hereby incorporated by reference. Said Sequence Listing has been filed as an electronic document via the USPTO patent electronic filing system in ASCII formatted text. The electronic document, created on Mar. 29, 2024, is entitled “081029-1399948-003010US_ST25.txt”, and is 22,668 bytes in size.

### BACKGROUND

Complications in pregnancy, such as miscarriage, preterm labour and preterm birth, are a significant problem and may have long-lasting effects on both the mother and child. Such complications are not uncommon, with miscarriage occurring in about 25% of pregnancies and preterm labour occurring in between 5 and 10% of pregnancies. Preterm labour is the single largest cause of the death of babies under the age of five anywhere in the world and a major cause of both severe and mild handicap in those who survive. About one third of cases of preterm labour are preceded by preterm premature rupture of the membranes. In these cases, there is a high risk of early onset neonatal sepsis, which itself is a risk factor for later handicap.

Lactobacilli are gram positive rod-shaped bacteria that are a part of the microbial flora of the human gut, mouth, and vagina. Vaginal Lactobacilli are thought to play an important role in resistance to infection via production of lactic acid and acidification of the vagina or by production of other antimicrobial products, such as hydrogen peroxide H₂O₂. It has been shown that high levels of Lactobacillus crispatus during pregnancy is linked to a decreased frequency of miscarriage, preterm labour and preterm birth, whilst the presence of Lactobacillus iners has been shown to be linked with preterm birth.

These findings, along with the widespread belief that lactobacilli generally promote vaginal health, suggest that women should re-colonize the vagina with Lactobacillus to prevent or minimise the chances of pregnancy complications. Whilst there are a wide range of “over the counter” Lactobacillus spp. containing products targeted at vaginal health and being encouraged to be used by pregnant women, there is little evidence of either colonisation or benefit when these products are used in isolation. See Yang et al., Effect of Oral Probiotic Lactobacillus rhamnosus GR-1 and Lactobacillus reuteri RC-14 on the Vaginal Microbiota, Cytokines and Chemokines in Pregnant Women Nutrients 2020, 12, 368; doi:10.3390/nu12020368. In Yang et al., the authors reported in Table 2, two incidents of preterm birth in the probiotic cohort and none in the placebo group. They conclude that they were unable to establish that the Lactobacillus probiotic treatment had an effect on PTB (See page 12).

Contrary to Yang et al., this invention affords a dramatic reduction in preterm birth within a cohort at risk for PTB. Moreover, the invention surprisingly teaches that the use of antibiotics as a pretreatment to reduce competition prior to administration of live therapeutics is not necessary when a high potency strain of L. crispatus in a high viability formulation is administered. This is important because the use of antibiotics to promote a healthy vaginal microbiome and reduce undesired bacteria is contraindicated for pregnant women or for pregnant women with asymptomatic bacterial vaginosis (BV). Bacterial vaginosis, or dysbiosis of the vaginal microbiome, has been associated with obstetric complications, including PTB. While BV is often asymptomatic, the CDC and ACOG recommend treatment with antibiotics only for symptomatic cases.

The conventional wisdom teaches that optimal therapeutic colonization of Lactobacillus in the vagina requires pretreatment with antibiotics. (See Bohbot et al., Efficacy and safety of vaginally administered lyophilized Lactobacillus crispatus IP 174178 in the prevention of bacterial vaginosis recurrence. J Gynecol Obstet Hum Reprod 2018; 47:81-86; Haahr T, Freiesleben N C, et al., Effect of clindamycin and a live biotherapeutic on the reproductive outcomes of IVF patients with abnormal vaginal microbiota: protocol for a double-blind, placebo-controlled multicentre trial. BMJ Open. 2020 Oct. 13; 10(10): e035866; and Heczko P B, et al. Supplementation of standard antibiotic therapy with oral probiotics for bacterial vaginosis and aerobic vaginitis: A randomised, doubleblind, placebo-controlled trial. BMC Womens Health 2015; 15:115).

There is a continued need for additional improved therapies aimed at increasing the probability of full term birth in pregnant subjects.

### SUMMARY OF INVENTION

The present invention relates to the use of a particular species of Lactobacillus having desirable characteristics suitable for use in pregnant women. It is a surprising finding of the inventors that the Lactobacillus composition herein disclosed can be used to supplement or replace the natural vaginal microbiota of a pregnant subject, resulting in protective outcomes against numerous pregnancy complications, without the need for prior or concomitant supportive antibiotic treatment.

In a first aspect, the present invention provides for a composition comprising L. crispatus cells for use in increasing the probability of full term birth in a pregnant female subject, wherein the composition is for topical vaginal application, wherein the L. crispatus cells functionally express a pullulanase gene to utilize glycogen and produce both L- and D-lactic acid, and wherein the subject has not been pretreated with an antibiotic, in particular an antibiotic targeting vaginal bacteria.

### FIGURES

FIGS. 1A and 1B show clinical protocol schematics for a pregnant subject receiving the composition herein disclosed. Women at high-risk of preterm birth recruited for LACTIN-V therapy were sampled at 14 weeks of gestation (T0), at the end of the loading dose (T1), during (T2) and at the end (T3) of the maintenance dosing phase, 28 weeks (T4), 36 weeks (T5) and at the time of delivery (T6).

FIG. 2 shows a pie chart displaying the proportion of risk factors for the 61 total active participants partaking in the clinical protocol herein disclosed.

FIG. 3 shows a pie chart displaying the proportion of ethnic origins for the 61 total active participants partaking in the clinical protocol herein disclosed.

FIG. 4 shows the outcomes of the 61 total active participants partaking in the clinical protocol herein disclosed.

FIG. 5 shows metataxonomic profiling of pregnant women receiving LACTIN-V therapy. Vaginal samples were collected from women at specific time points during pregnancy and the bacterial composition characterized by Illumina MiSeq based amplicon sequencing of the V1-V2 hypervariable regions of bacterial 16S rRNA genes. (A) Mean raw and log-transformed sequence read counts mapped to L. crispatus were significantly increased after the loading phase and remained higher throughout pregnancy (ANOVA, p<0.001, FDR q<0.001). This equated to a shift in the prevalence of L. crispatus-dominated vaginal microbiota from 35% pre-LACTIN-V therapy, to 95% post-loading dose. (B) In contrast, mean raw and log-transformed sequence read counts mapped to L. iners were significantly decreased after the loading dose (ANOVA, p<0.001, FDR q<0.001). T0=14 weeks of gestation, T1=15 weeks of gestation (at end of the loading dose), T2=18 weeks (during maintenance dosing phase) and at the end (T3) of the maintenance dosing phase, 28 weeks (T4), 36 weeks (T5) and at the time of delivery (T6).

### DETAILED DESCRIPTION

The following description is presented to enable any person skilled in the art to make and use the invention. Various modifications to the disclosed embodiments will be readily apparent to those skilled in the art.

In order that the present invention may be more readily understood, certain terms are first defined. Additional definitions are set forth throughout the detailed description.

As used herein, the terms “Lactobacillus crispatus” and “L. crispatus” are used interchangeably and refer to a species of the Lactobacillus genus. The species is generally distinguished from other lactobacilli based on the polynucleotide sequence of the 16S ribosomal RNA gene. L. crispatus is one of a number of Lactobacillus species which is a vaginal species capable of producing hydrogen peroxide, and both L- and D-lactic acid. The terms “Lactobacillus crispatus” and “L. crispatus” additionally refer to any strain having at least 97% sequence homology to the 16S ribosomal RNA gene sequence of the 16S ribosomal RNA gene of L. crispatus.

As used herein, the term “full term birth” refers to a term of pregnancy wherein the baby is born on or after 37 weeks. Accordingly, the term “increasing the probability of full term birth” may be interchangeable with the terms “increasing the probability of preventing preterm labour or preterm delivery”, wherein “preterm labour” and “preterm delivery” refer to a term of pregnancy wherein the baby is born before 37 weeks. It is also intended to encompass increasing the probability of full term birth by reducing the risk of miscarriage in a subject, where the term “miscarriage” is defined as a spontaneous loss of pregnancy up to 24 weeks.

As used herein, the term “topical” and “topical application” are used interchangeably and refers to a composition that is applied to a particular place on or in the body, most commonly to the skin or mucosal membranes. Accordingly, the term “topical” may refer to a composition that is applied directly to the subject. In the present invention, this may refer to application of the composition herein described to the vaginal or cervical epithelium of the pregnant subject.

As used herein, the term “pretreatment”, in the context of the present invention, refers to the subject not having any kind of antibiotic treatment in advance, or simultaneously, as having the composition herein disclosed topically administered to the vagina of said subject.

As used herein, the term “lyophilised” or “lyophilised powder” are used interchangeably and refer to the process by which the powder is produced via freezing a substance and then reducing the concentration of water, by sublimination and/or evaporation to levels which do not support biological or chemical reactions.

As used herein, the term “viable” and “viable cells” are used interchangeably and refer to L. crispatus cells that are able to grow and reproduce. As used herein, the term “potency” refers to the number of viable microbial cells delivered per medicant unit (i.e. lyophilized powder). For the composition herein disclosed to be efficacious in vivo, both colonisation of the vaginal epithelial cells by the microbial cells at a potency of at least 10⁸ CFU per medicant unit and a biological effect are necessary.

As used herein, the term “MRS medium”, “De Man, Rogosa and Sharpe agar” and “MRS” refer to a selective culture medium designed to favour the growth of L. crispatus cells. Such culture medium will be known to the skilled person and is commercially available. Typically, the MRS medium comprises 10 g Bacto Proteose Peptone No. 3, 10 g Bacto Beef Extract, 5 g Bacto Yeast Extract, 20 g Bacto Dextrose Extract, 1 g Tween 80, 2 g ammonium citrate, 5 g sodium acetate, 0.1 g magnesium sulfate, 0.05 g manganese sulfate, and 2 g dipotassium phosphate, brought up to 1000 ml with reagent grade H₂O. For MRS agar, 10 g of agar may be added to the mixture. The medium may be adjusted to pH 6.5±0.2 at 25° C. for optimum conditions, although any conditions that favour the growth of the L. crispatus cells may also be used. In some instances, a MRS agar may be utilised to optimise growth conditions for a specific L. crispatus strain, for example, the L. crispatus SJ-3C strain or L. crispatus strain CTV-05. The SJ-3C strain was deposited with the American Type Cell Culture, 10801 University Blvd., Manassas, Va. 20110-2209 on Jun. 23, 2009, and granted accession number PTA-10138.

This deposit was made in accordance with the Budapest Treaty and as described in 37 CFR 1.801-1.809. The CTV-05 strain was deposited on 20 Apr. 1999 under American Type Culture Collection (ATCC) accession number 202225, deposited by GyneLogix, Inc., acquired by Osel, Inc. on Feb. 28, 2003.

As used herein, the terms “first trimester”, “primary trimester” and “initial trimester” are used interchangeably and refers to the period of pregnancy beginning on the first day of the subject's last period and lasts until the last day of week 12 of the pregnancy. The terms “second trimester” and “middle trimester” are used interchangeably and refers to the period of pregnancy beginning on the first day of week 13 of the pregnancy, and lasting till the last day of week 27 of the pregnancy.

As used herein, the terms “last menstrual period” and “LMP” are used interchangeably and refers to the first day of the subject's last menstrual period before falling pregnant. The LMP is commonly used to calculate the expected date or due date of the delivery of the baby.

As used herein, the term “third trimester” or “final trimester” are used interchangeably and refers to the period of pregnancy beginning on the first day of week 27 or week 28 of the pregnancy, and lasting till the end of the pregnancy.

As used herein, the term “total bacterial population” refers to the total number of bacterial microorganisms that colonize the vagina, i.e. contribute to the make up the “vaginal microbiota” or “vaginal microbiome”. This may include numerous species of microorganisms (including fungal and viral species) and includes both viable and non-viable microorganism.

As used herein, the term “a short cervix” refers to a subject who has a cervix of 25 mm or less in length as measured by conventional means, for example, an ultrasound, and thus increases the risk of pregnancy loss, preterm labor and/or preterm birth in that particular subject. A short cervix as defined herein may be 15-25 mm in length, 16-25 mm in length, 17-25 mm in length, 18-25 mm in length, 19-25 mm in length, 20-25 mm in length, 21-25 mm in length, 22-25 mm in length, 23-25 mm in length or 24-25 mm in length.

As used herein, the term “Lactobacillus iners” and “L. iners” are used interchangeably and refers to an additional species of the Lactobacillus genus.

As used herein, the term “daily” refers to the composition herein disclosed being administered to a subject on a daily basis. The number of times the composition is administered in one day may be multiple times. For example, the composition herein disclosed may be administered 1 or 2 times a day, preferably, the composition is administered 1 time a day. Said composition may be administered at any time throughout the day, however, it is envisaged that the optimal time for administration is at bedtime, preferably prior to lying down to sleep.

As used herein, the term “dysbiosis” or “vaginal dysbiosis” are used interchangeably and refer to the displacement of optimal vaginal microbiota by diverse bacteria.

As used herein, the terms “effective amount” or “pharmaceutically effective amount” refer to a sufficient amount of an agent to provide the desired biological or therapeutic result. In the context of the present invention, these terms may refer to the amount of L. crispatus cells required to achieve the desired effect, for example, increasing the probability of full term birth in pregnant females.

As used herein, the term “loading period” refers to the period of time at the beginning of dosing, when a subject may receive multiple doses of the composition herein disclosed within a defined period, for example, 2-7 days.

As used herein, the “pullulanase gene positive” refers to strains of L. crispatus that express the glycogen debranching enzyme pullulanase, allowing them to utilize glycogen (see Pan et al., Host and body site-specific adaptation of Lactobacillus crispatus genomes. NAR Genomics and Bioinformatics, 2020: 2(1); Van Der Veer et al., Comparative genomics of human Lactobacillus crispatus isolates reveals genes for glycosylation and glycogen degradation; implications for in vivo dominance of the vaginal microbiota). The L. crispatus strain SJ-3C pullulanase may have an amino acid sequence with at least 95% homology to SEQ ID NO: 1. The L. crispatus strain CTV-05 pullulanase may have an amino acid sequence with at least 95% homology to SEQ ID NO: 2.

As used herein, the term “water activity” and the notation “a_{w}” refer to the amount of unbound water in a sample. Water activity is a thermodynamic measure of water expressed as the vapor pressure of water in a sample divided by the vapor pressure of pure water at a given temperature. Water activity and a_{w} are defined to be equal to the Equilibrium Relative Humidity (“ERH”) divided by 100. ERH is the equilibrium state at which the dry powder neither absorbs nor loses moisture to the environment. The ERH is influenced by the composition of all ingredients, particularly those with high water content, which may be present as free or bound water. The amount of free water can influence the storage stability and purity of the dry powder which could result in undesired degradation of activity or growth of contaminating microorganisms during storage.

As used herein, the term “wet weight” refers to the weight (grams) of the cell pellet following centrifugation and decantation of the supernatant. In general, following the step of cell harvesting, centrifuge bottles are pre-weighed, cells are spun down, the supernatant is decanted, and the bottles are weighed again. The difference in weight is the wet weight of the pellet.

It is believed that the link between a vaginal microbiota deplete in Lactobacillus, or rich in Lactobacillus iners, and preterm birth is via activation of inflammatory pathways within the vaginal environment. Without being bound by theory, it is thought that a vaginal microbiota rich in L. crispatus is protective against this inflammatory activation and may contribute to enhanced rates of full term birth in pregnant subjects.

It is a surprising effect of the present invention that vaginal administration of L. crispatus can produce the advantageous effects herein described without the need for any concomitant antibiotic treatment to support L. crispatus colonisation.

Accordingly, in a first aspect, the present invention provides a composition comprising L. crispatus cells for use in increasing the probability of full term birth in a pregnant female subject, wherein the composition is for topical vaginal application, wherein the L. crispatus cells functionally express a pullulanase gene to utilize glycogen and produce both L- and D-lactic acid, and wherein the subject has not been pretreated with an antibiotic. The composition herein disclosed may also be used for reducing the probability of spontaneous preterm birth <34 weeks gestation.

The composition herein disclosed is particularly aimed at pregnant subjects who satisfy the following criteria:
i) subjects who have a prior history of preterm birth or short cervix of 25 mm or less in length; and/or
ii) subjects who have a L. iners population of at least 50% in a vaginal fluid sample; and/or
iii) subjects who have dysbiosis with less than 50% Lactobacillus spp. in a vaginal fluid sample; and/or
iv) subjects who have not been pretreated with an antibiotic targeting vaginal bacteria.

The strains of L. crispatus useful in this invention functionally express a pullulanase gene (glgX) which confers the ability to utilize glycogen which is an important carbon source for some vaginal strains of L. crispatus. Glgx encodes a type I pullulanase debranching enzyme (LKBG_01742). Glgx can be identified by amplification of internal fragment of LBKG_01742 (982 bp, using the following primer pair:

AM51:
(SEQ ID NO: 3)
CGGTCCTTATGTTCGTTCGA,
and
AM52:
(SEQ ID NO: 4)
CACCTGGAGTGGTTGGACTT.

The ability to utilize glycogen can be determined phenotypically by conventional test kits such as the carbohydrate utilization test kit from Biomerieux, https://www.biomerieux-usa.com/sites/subsidiary_us/files/18_api-ref-guide_v7.pdf, specifically kit API 50 CH Cat #50300 and #50410.

The L. crispatus cells may also have a H₂O₂ positive phenotype. The H₂O₂ phenotype is associated with sustained vaginal colonization (Vallor, A. C., et al., J Infect Dis. 2001 Dec. 1; 184 (11): 1431-6) and immunomodulatory effects in the vagina (Mitchell, C. et al. Sex Transm Dis Sex Transm Dis. 2015 July; 42 (7): 358-363. Additionally, production of lactic acid, specifically L- and D-lactic acid, by the Lactobacillus spp has been shown to inhibit the growth of pathogens in vitro.

The L. crispatus cells of the present invention may produce H₂O₂. H₂O₂ production by L. crispatus can be quantitated by any means for measuring H₂O₂. Methods used to measure H₂O₂ production are well known in the art, and can include the culture method or the direct detection method. The culture method can involve measuring H₂O₂ production by quantifying the intensity of a blue pigment formed when the L. crispatus cells are inoculated onto tetramethylbenzidine medium (TMB) and incubated under anaerobic conditions. For example, L. crispatus may be incubated on a TMB agar plate for about 48 hours under anaerobic conditions at 37° C. The agar plate is then exposed to ambient air. Exposure to the ambient air causes the H₂O₂ produced to react with the horseradish peroxide in the agar to oxidise the TMB, causing L. crispatus colonies to turn blue. On this basis, the strain is scored 0 as a non-producer, 1 as a weak producer, 2 as a moderate producer, or 3 as a strong producer. A strong H₂O₂ producing L. crispatus strain will turn blue within 10 min of exposure to air, and dark blue by 30 min (Pendharkar, S. et al. BMC Infect Dis 2013 January 13:43. doi: 10.1186/1471-2334-13-43). Alternatively, the direct detection method may be used to measure the quantity of H₂O₂ between 0 and 100 mg/L using commercially available H₂O₂ detection strips (e.g., available from MilliporeSigma).

The L. crispatus cells of the present invention produce both L- and D-lactic acid, the production of which has been shown to inhibit the growth of pathogens in vitro. Preferably, the L. crispatus cells of the present invention produces at least about 0.75 mg/100 ml lactic acid, more preferably at least about 4 mg/100 ml lactic acid, and even more preferably at least about 8.8 mg/100 ml lactic acid under effective growth conditions. Simple, direct and automation-ready procedures for measuring lactate concentration are available see: BioAssay Systems' EnzyChrom™ lactate assay kit based on lactate dehydrogenase catalyzed oxidation of lactate, in which the formed NADH reduces a formazan (MTT) Reagent. The intensity of the product color, measured at 565 nm, is proportionate to the lactate concentration in the sample. Another means for measuring lactic acid is using HPLC.

A further characteristic of the L. crispatus cells may be said L. crispatus cells having a percent vaginal epithelial cell (VEC) cohesion value of at least about 50%. A “percent VEC cohesion value” is defined as the percentage of VECs to which at least one L. crispatus cell is adhered in the total number of VECs in an identified group. According to the present invention, the terms “cohesion” and “adherence” can be used interchangeably. Adherence of microbial cells to vaginal epithelial cells is critical for colonization and biological effect. Successful adherence of a L. crispatus cell of the medical powder to a vaginal epithelial cell results in successful colonization of the vaginal epithelial cell. Long term in vivo colonization is a goal of the products and methods of the present invention, and “percent VEC cohesion value” is a good predictor of whether a significant number of VECs will accept microbial cells in vitro and in vivo. Methods used to determine acceptable VEC cohesion values are well known in the art and can be found in U.S. Pat. Nos. 6,468,526 and 6,093,394. See also Kwok, et al., J. Urol. 2006, 176:2050-2054.

Yet a further characteristic of the L. crispatus cells used herein may be said L. crispatus cells having genetic stability over time, both in vivo and in vitro. According to the present invention, “genetic stability” refers to the ability of successive generations of a Lactobacillus strain to substantially maintain the identical genetic profile of the mother strain. In other words, successive generations of a genetically stable strain will not acquire substantial mutations in its genomic DNA over a period of time. Repetitive Sequence Polymerase Chain Reaction (Rep PCR) can be used to confirm genetic identity and stability of a strain of Lactobacillus over time after either in vitro storage or in vivo colonization of vaginal epithelial cells. Rep PCR methods used to confirm genetic identity and stability Lactobacillus strains are well known in the art and can be found in U.S. Pat. No. 6,093,394. See also, Antonio & Hillier, J. Clin. Microbiol. 2003, 41:1881-1887.

As referenced herein, the antibiotic to be avoided is an antibiotic targeting vaginal bacteria, wherein the vaginal bacteria may be associated with bacterial vaginosis (BV), for example, Gardnerella vaginalis, Atopobium vaginae and other diverse anaerobic bacteria, such as Prevotella spp. and Mobiluncus spp., or associated with sexually transmitted infections, such as Chlamydia trachomitis and Trichomonas vaginalis. As used herein, the term “antibiotic targeting vaginal bacteria” refers to any antibiotic which targets these, or any other, bacteria residing in the vagina. Commonly, such bacterial infections of the vagina are treated with antibiotics such as clindamycin, metronidazole, tinidazole, and/or secnidazole. It is a surprising advantage of the present invention that the composition herein disclosed may be effective in increasing the probability of a pregnant subject reaching full term, without the need for additional therapies aimed at reducing or killing undesirable bacteria populations, such as antibiotics or bacteriophage therapies.

The composition for use herein disclosed may be a combination of a lyophilised powder comprising the living bacteria formally called a drug substance. The drug substance is then combined with excipients such as maltodextrin to yield the final medicament formally called the drug product. Accordingly, as used herein, the terms “composition” and “drug product” are interchangeable. Such a formulation allows for a final dosage form that has a longer shelf life, enhanced stability and fewer restrictions on transportation and storage when compared to alternative formulations. The skilled person will be aware of suitable methods of preparing such a lyophilised powder and the appropriate equipment, for example, a refrigeration system, a vacuum system, a control system, a product chamber or manifold and a condenser.

The composition or powder herein disclosed may further comprise an excipient or pharmaceutically acceptable carrier. Accordingly, the powder herein disclosed may further comprise an excipient or pharmaceutically acceptable carrier. As used herein, the terms “excipient” and “pharmaceutically acceptable carrier/excipient” are used interchangeably and refer to inert substances formulated alongside the active ingredient of the formulation, i.e. the L. crispatus cells. They are commonly included to enhance stability or to confer a therapeutic enhancement on the active ingredient in the final dosage form, for example, facilitating drug absorption, reducing viscosity or enhancing solubility. Examples of suitable excipients and/or pharmaceutically acceptable carriers include, but are not limited to, maltodextrin, fructooligosaccharide, galactooligosaccharide, starch, pre-gelatinized starch, microcrystalline cellulose, calcium carbonate, dicalcium phosphate, colloidal SiO₂, Pharmasperse®, mannitol, trehalose, xylitol, sodium ascorbate, lactose, sucrose, polyvinyl, pyrrolidone, crosspovidone, glycine, magnesium stearate, sodium stearyl fumarate, cyclodextrins and derivatives and mixtures thereof.

In a specific embodiment, the composition or powder herein disclosed may further comprise trehalose, xylitol, sodium ascorbate, colloidal silicon dioxide and maltodextrin in addition to the L. crispatus strain, for example, L. crispatus CTV-05 or L. crispatus SJ-3C.

The composition or powder herein disclosed may be diluted with an excipient between 3-fold and 10-fold. The composition or powder herein disclosed may be combined with an excipient at a ratio of composition/powder to excipient between 1:1 and 1:12 w/w. The composition or powder herein disclosed may be combined with an excipient at a ratio of composition/powder to an excipient of 1:1, 1:2, 1:3, 1:4, 1:5, 1:6, 1:7, 1:8, 1:9, 1:10, 1:11, or 1:12 w/w. Preferably, the composition or powder herein disclosed may be combined with an excipient at a ratio of composition/powder to an excipient of between 1:1 and 1:10 w/w. More preferably, the composition or powder herein disclosed may be combined with an excipient at a ratio of composition/powder to an excipient of between 1:1 and 1:5 w/w. Even more preferably, the composition or powder herein disclosed may be combined with an excipient at a ratio of composition/powder to an excipient of between 1:1 and 1:3 w/w.

In a specific embodiment, the composition/powder herein disclosed may be combined with maltodextrin at a ratio of composition/powder to maltodextrin of between 1:1 and 1:10 w/w. The composition/powder herein disclosed may be combined with maltodextrin at a ratio of composition/powder to maltodextrin of between 1:1 and 1:5 w/w. The composition/powder herein disclosed may be combined with maltodextrin at a ratio of composition/powder to maltodextrin of between 1:1 and 1:3 w/w.

In a specific embodiment, the composition/powder herein disclosed may be combined with fructooligosaccharide at a ratio of composition/powder to fructooligosaccharide of between 1:1 and 1:10 w/w. The composition/powder herein disclosed may be combined with fructooligosaccharide at a ratio of composition/powder to fructooligosaccharide of between 1:1 and 1:5 w/w. The composition/powder herein disclosed may be combined with fructooligosaccharide at a ratio of composition/powder to fructooligosaccharide of between 1:1 and 1:3 w/w.

In a specific embodiment, the composition/powder herein disclosed may be combined with galactooligosaccharide at a ratio of composition/powder to galactooligosaccharide of between 1:1 and 1:10 w/w. The composition/powder herein disclosed may be combined with galactooligosaccharide at a ratio of composition/powder to galactooligosaccharide of between 1:1 and 1:5 w/w. The composition/powder herein disclosed may be combined with galactooligosaccharide at a ratio of composition/powder to galactooligosaccharide of between 1:1 and 1:3 w/w.

The composition for use herein disclosed may have a particle size between 800 and 100 μm generated by conventional sieving methodology using sieves of 20 to 200 mesh.

The composition for use herein disclosed may produce an amount of viable cells between 1×10⁸ and 1×10¹⁰ CFU per dose when plated on MRS agar, Rogosa or a combination of the two, and preferably between 5×10⁸ to 5×10⁹ CFU per dose. In a preferred embodiment, a high potency strain of L. crispatus is used. Such a strain, in combination with the high viability formulation herein disclosed, provides for clinically significant results in relation to increasing the probability of a pregnant female reaching full term, or equally, increasing the probability of preventing preterm labour or preterm delivery in a pregnant female.

As an alternative to MRS medium, any other medium that provides effective growth of the L. crispatus cells, without the loss of the desired functional characteristics, may also be used. Preferably, such a medium may include a source of assimilable organic carbo, a source of assimilable nitrogen and appropriate salts and trace metals.

The amount of viable cells may be between 1×10⁸ and 1×10¹⁰, 2×10⁸ and 1×10¹⁰, 3×10⁸ and 4×10¹⁰, 5×10⁸ and 1×10¹⁰, 6×10⁸ and 1×10¹⁰, 7×10⁸ and 1×10¹⁰, 8×10⁸ and 1×10¹⁰, 9×10⁸ and 1×10¹⁰, 1×10⁸ and 1×10⁹, 2×10⁸ and 1×10⁹, 3×10⁸ and 1×10⁹, 4×10⁸ and 1×10⁹, 5×10⁸ and 1×10⁹, 6×10⁸ and 1×10⁹, 7×10⁸ and 1×10⁹, 8×10⁸ and 1×10⁹, 9×10⁸ and 1×10⁹, 1×10⁹ and 1×10¹⁰, 2×10⁹ and 1×10¹⁰, 3×10⁹ and 1×10¹⁰, 4×10⁹ and 1×10¹⁰, 5×10⁹ and 1×10¹⁰, 6×10⁹ and 1×10¹⁰, 7×10⁹ and 8×10¹⁰, 9×10⁹ and 1×10¹⁰. Preferably, the amount of viable cells may be between 5×10⁸ and 5×10⁹ CFU per dose.

The composition or powder herein disclosed may be packaged in dosages of between about 100 mg and 600 mg, for example, of about 100 mg, of about 150 mg, of about 150 mg, of about 200 mg, of about 250 mg, of about 300 mg, of about 350 mg, of about 400 mg, of about 450 mg, of about 500 mg, of about 550 mg, or of about 600 mg. The composition/powder herein disclosed may be packaged in dosages of between about 150 mg and 450 mg, or about 150 mg and about 400 mg, or about 150 mg and about 350 mg. Preferably, the composition/powder herein disclosed may be packaged in dosages of about 150 mg to 250 mg, more preferably in dosages of about 200 mg.

The composition or powder herein disclosed may be topically administered to the vagina using a pre-filled vaginal applicator. In a specific embodiment, the composition is a powder formulation of L. crispatus strain CTV-05 provided in a pre-filled vaginal applicator at a dose of 2×10⁹ in 200 mg. In another specific embodiment, the composition is a powder formulation of L. crispatus strain SJ-3C provided in a pre-filled vaginal applicator at a dose of 2×10⁹ in 200 mg. In some embodiments, the vaginal applicator and powder formulation are provided separately. In these instances, the vaginal applicator is filled with the powder formulation prior to use by the intended user. It is believed that this application method is particularly efficient at delivering the composition or powder herein disclosed, allowing for colonisation of the vagina by said L. crispatus strain and replenishment of depleted L. crispatus populations.

The composition for use herein disclosed may be for use in a subject who is planning a pregnancy, or is in the first or second trimester.

In a preferred embodiment, the subject is early in the first trimester. The subject may be between 10-16 weeks after their last menstrual period (LMP). In an alternative preferred embodiment, the subject is early in the second trimester.

The subject may be between 10-16 weeks, 11-16 weeks, 12-16 weeks, 13-16 weeks, 14-16 weeks, 15-16 weeks, 10-11 weeks, 10-12 weeks, 10-13 weeks, 10-14 weeks, 10-15 weeks, 11-12 weeks, 11-13 weeks, 11-14 weeks, 11-15 weeks, 12-13 weeks, 12-14 weeks, 12-15 weeks, 13-14 weeks, 13-15 weeks or 14-15 weeks after their LMP.

In an alternative embodiment, the composition may be for administration to the subject up to 34 weeks after last menstrual period. Accordingly, the composition herein disclosed may also be suitable for administration to subjects who are in the third trimester of pregnancy.

The composition for use herein disclosed may be for use in a subject who has a prior history of preterm birth; and/or a short cervix of 25 mm or less in length. The composition for use herein disclosed may also be for use in a subject who has a prior history of miscarriage(s) or who is at higher risk of miscarriage for other reasons.

It is known that the shorter the cervix of the pregnant subject, the higher the risk of that subject experiencing complications. As such, the use of the composition herein disclosed in these particular subjects is envisaged as being particularly advantageous. The length of the cervix may be quantified using techniques known to those in the art, for example, a transvaginal ultrasound.

Additional risk factors for miscarriage, preterm labor and/or preterm birth include, but are not limited to, prior history of preterm labor and/or preterm birth, prior LLETZ (large loop excision of the transformation zone), treatment for cervical intraepithelial neoplastia, being pregnant with twins or triplets (multiple gestations), use of reproductive technology (e.g. in vitro fertilization), reproductive organ abnormalities, urinary tract infections, sexually transmitted infections, vaginal bacterial infections (e.g. bacterial vaginosis and trichomoniasis), high blood pressure, abnormal vaginal bleeding, being underweight or overweight/obese, short time between pregnancies, placenta previa, being at risk of rupture of the uterus, diabetes and gestational diabetes, blood clotting, ethnicity, age, various lifestyle/environmental factors (e.g. smoking, drinking, stress levels, exposure to environmental pollutants and drug abuse), and any combinations thereof. The use of the composition herein disclosed in subjects who have the above risk factors are also envisaged as being particularly advantageous.

Where the subject suffers from certain high risk factors, for example, a shortened cervix, the subject may use the composition herein disclosed in combination with other standard therapies, for example, cervical cerclage, progesterone therapy or antenatal corticosteroid therapy. Suitable therapies, and to which risk factor they apply, will be well known by those skilled in the art.

The composition for use herein disclosed may be used wherein a vaginal fluid sample obtained from the subject has a total bacterial population, and at least 50% of the total bacterial population is determined to comprise Lactobacillus iners. This particular species of Lactobacillus is known to have an increased risk of shortening of the cervix, which may subsequently lead to the need to insert a cervical stitch, and therefore a possible increased risk of preterm birth. As such, pregnant subjects who have particularly high levels of this specific species of Lactobacillus are envisaged as particularly benefitting from the invention herein disclosed.

The total bacterial population may be quantified using techniques known to the skilled person, for example, qPCR, microarray and next generation sequencing techniques. Whole genomic DNA may be extracted and amplified using the barcoded universal primers and deep sequenced using the Illumina Miseq pyrosequencing platform, and subsequently analysed using the Michigan Ribosome Database Project.

The vaginal fluid sample may be collected via a sterile cotton swab being inserted into the vagina of the pregnant subject, or by any other suitable means. The sample may be collected from the posterior vaginal fornix.

The subject may have a total bacterial population comprising at least 50% of L. iners. For example, the subject may have a total bacterial population comprising at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% L. iners. It is envisaged the use of the composition herein disclosed in these subjects will allow for the replacement of L. iners colonies with that of L. crispatus, and thus provide a more protective environment, which in turn is expected to increase the probability of pregnant subjects reaching full term.

Alternatively, the composition for use herein disclosed may be used wherein a vaginal fluid sample obtained from the subject has a total bacterial population, and wherein less than 50% of the total bacterial population comprises Lactobacillus spp. Specifically, the subject may have a total bacterial population wherein less than 50% of the total bacterial population are Lactobacillus spp. associated with resisting infection and producing antimicrobial products, such as H₂O₂ and lactic acid. Examples of said Lactobacillus species include, but are not limited to L. crispatus, L. jensenii and L. gasseri.

The subject may have a total bacterial population comprising less than 50% of Lactobacillus spp., for example, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1% or do not comprise a Lactobacillus spp. at all. The subject may have a total bacterial population wherein less than 50% of the total bacterial population are Lactobacillus spp. associated with resisting infection and producing antimicrobial products, for example, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1% or do not comprise a Lactobacillus spp. at all. The composition herein disclosed is therefore intended to supplement existing advantageous Lactobacillus species, or replace (either partially or fully) non-advantageous Lactobacillus species, for example, L. iners.

In a specific embodiment, the composition for use herein disclosed provides a significant increase in the prevalence of L. crispatus-dominated vaginal microbiota from pre-LACTIN-V therapy to post-loading dose, the increase in which corresponds to a significant decrease in the prevalence of L. iners. Further, these microbiota changes are sustained well after the last dose of LACTIN-V.

The L. crispatus strains suitable for use in the present invention may be any L. crispatus strain wherein the L. crispatus cells produce H₂O₂, are pullulanase gene positive, utilize glycogen, and produce both L- and D-lactic acid. The L. crispatus strains suitable for use in the present invention may also display the following characteristics; self-aggregation, antagonism of urogenital pathogens, for example, uropathogenic Escherichia coli, Candida albicans, Staphylococcus aureaus, Streptococcus agalactiae group B and Gardnerella vaginalis, demonstrate good growth in culture and good viability after drying. Accordingly, suitable strains for use in the present invention may be detected and isolated from natural sources using appropriate screening techniques that are known in the art to identify the aforementioned desirable characteristics.

In a preferred embodiment, the L. crispatus may be L. crispatus strain CTV-05, deposited on 20 Apr. 1999 under American Type Culture Collection (ATCC) accession number 202225, deposited by GyneLogix, Inc., acquired by Osel, Inc. on Feb. 28, 2003. In an alternative preferred embodiment, the L. crispatus may be L. crispatus strain SJ-3C, deposited on Jun. 23, 2009 under ATCC accession number PTA-10138, deposited by Osel, Inc. In yet a further alternative embodiment, the L. crispatus strains may be L. crispatus strains MV-3A-US and/or MV-1A-US, said strains available through BEI Resources (NIH/NIAID). Combinations of strains may also be used in a complementary manner to broaden the functionality of the present invention where appropriate, for example, where a woman presents with different community state types (CSTs). As used herein, the terms “community state types” and “CSTs” refer to various distinct vaginal microbiomes that exist and well known to the skilled person.

Mutated versions of the aforementioned strains, or any other suitable strain according to the invention, for purposes of enhancing desirable characteristics for example, are also suitable for the purpose of the invention herein disclosed. The term “mutated” herein refers to a L. crispatus strain in which the nucleotide composition of the strain has been modified by mutation(s) that occur naturally or that are the results of genetic engineering or selection. Methods of introducing mutations into bacterial strains are well known in the art and may include subjecting said strain to at least one round of chemical and/or radiation mutagenesis or using genetic engineering techniques, such as CRISPR or homologous recombination.

Methods used to differentiate between L. crispatus strains include, but are not limited to Rep-PCR, as described in Antonio & Hillier, J. Clin. Microbiol. 2003, 41:1881-1887, multilocus sequence typing (MLST), originally developed to identify strains of pathogens (see, e.g., Maiden, M. C., et. al 1998, Multilocus sequence typing: a portable approach to the identification of clones within populations of pathogenic microorganisms. Proc. Natl. Acad. Sci. USA., 95:3140-2145), whole genome sequencing and qPCR can be used to quantify L. crispatus.

The composition for use herein disclosed may be for administration to a subject who has previously been administered the composition for use. The composition for use herein disclosed may be for administration to a subject who has previously been administered the powder for use herein disclosed.

Accordingly, the present invention provides for multiples doses of the composition herein disclosed. The skilled person will recognise that the precise number of times the composition herein disclosed is to be administered to a pregnant subject will depend on a variety of factors, for example, the pregnancy term, total bacterial population results and risk factors. It is envisaged that subjects determined to have high levels of L. iners (>50% prevalence of total bacteria) and/or low levels of other advantageous Lactobacillus species, or deemed to be particularly high risk, may benefit from more frequent administration of the composition herein disclosed than other subjects not in those groups.

Accordingly, the present invention provides for a method of increasing the probability of full term birth in a pregnant female subject, wherein the composition herein disclosed is for administration to a subject who has previously been administered the composition herein disclosed.

The composition for use herein disclosed may be for administration daily for 2 to 7 days and then once or twice weekly thereafter.

The composition for use herein disclosed may be for administration daily for 2, 3, 4, 5, 6 or 7 days. Alternatively, the composition for use herein disclosed may be for administration daily between 2 and 7 days, or 3 and 6 days, or 4 and 5 days, or 4 and 7 days. Preferably, the composition herein disclosed is administered daily for between 2 and 7 days, yet more preferably for 5 or 7 days.

Following administration of the composition daily for 2 to 7 days, the subject may further receive the composition once or twice weekly thereafter up to the end of the pregnancy. In a preferred embodiment, the composition is not administered to the subject past 34 weeks. In yet a further preferred embodiment, the composition is administered once weekly (single dose) for a further 6 weeks. Where the composition is to be administered to the subject twice a week, the second administration may be 3-4 days following the first administration of the composition. In a specific embodiment, the subject may be administered a “loading phase” of 5 daily doses of the composition or powder herein disclosed, followed by a “maintenance phase” of 6 weekly doses of the composition or powder herein disclosed, for a total of 11 doses.

In a specific embodiment, the present invention provides for a method of increasing the probability of full term birth in a pregnant female subject having a predisposition of preterm birth, the method comprising the steps of:
i) selecting the pregnant female subject within the first or second trimester who has a prior history of preterm birth; or a short cervix of 25 mm or less in length; or has a Liners population of at least 50% in a vaginal fluid sample; or has dysbiosis with a Lactobacillus population of less than 50% in a vaginal fluid sample; and wherein the pregnant female subject has not been pretreated with an antibiotic targeting vaginal bacteria; and,
ii) vaginally administering multiple doses of lyophilized L. crispatus by direct application of L. crispatus to the vaginal mucosa in a powdered form; wherein the powdered form has an average size particle of less than 500 μm and produces an amount of viable cells between 1×10⁸ to 1×10¹⁰ CFU per dose when plated on MRS agar; and,
   wherein the L. crispatus cells produce H₂O₂; and both L- and D-lactic acid, and; wherein the dosing is in an amount effective to reduce the probability of preterm birth by increasing the vaginal population of L. crispatus.

Additional details of the methods used herein are found below, with further details found within U.S. Pat. No. 11,083,761 B2.

### Culturing Vaginal Bacteria

The L. crispatus strains useful for the present invention can be grown in liquid or on solid media (e.g., agar), preferably MRS agar. The L. crispatus cells are preferably cultured anaerobically or microaerophilic conditions and the temperature of the culture medium can be any temperature suitable for growth of L. crispatus cells. L. crispatus strains for the instant invention can be cultured in anaerobic conditions and are generally grown at about 37° C. Effective culture conditions for vaginal L. crispatus strains useful for the instant invention are well known in the art. Specific culture conditions, culture media and methods of culturing L. crispatus strains, e.g., U.S. Pat. Nos. 8,329,417, 6,093,394, and Davis, C. Enumeration of probiotic strains: Review of culture-dependent and alternative techniques to quantify viable bacteria. J Microbiol Methods. 2014; 103:9-17.

The culture medium is inoculated with an actively growing culture of the L. crispatus strain in an amount sufficient to produce, after a reasonable growth period, a suitable cell density (or potency) for producing the composition/powder herein disclosed. A non-limiting example of a reasonable growth period of the L. crispatus cells used herein is a generation time of between 1 to 2.5 hours. The cells are grown to a preferred cell density in the range of from about 10⁸ CFU/mL to about 10¹⁰ CFU/mL. A culture-based method is used to determine the cell density, in which serial dilutions of L. crispatus cultures are plated onto MRS agar plates and incubated for 48 hr anaerobically at 37° C. Colonies on the plates are counted and the number of CFUs (colony forming units) in the samples are calculated as CFU/mL or CFU/gram.

Once the cells are grown to preferred cell density, the bacterial cells can be harvested using any suitable method to remove the cells from the culture media. Non-limiting exemplary methods for harvesting the cultured cells includes, filtration, centrifugation, and sedimentation. In some examples, harvesting cultured cells can involve hollow fiber filtration and washing via diafiltration. Methods for harvesting cultured L. crispatus cells are well known in the art. After separation of the cells from the culture media and/or washing of the biomass, the cells to centrifuged to form a cell pellet in preparation for production of the composition/powder herein disclosed.

### Preparation of the Aqueous Preservation Medium

The bacterial cell pellet formed from the methods herein described may be a suitable aqueous preservation medium, where the weight ratio of cell pellet wet weight (grams) to preservation media (mL) can be between 1:1 and 1:8 grams of cell pellet to milliliter of preservation media. In some embodiments, the bacterial cell pellet is resuspended in a suitable aqueous preservation medium, where the weight ratio of cell pellet wet weight (grams) to preservation media (mL) can be between 1:1 and 1:7 grams of cell pellet to milliliter of preservation media, or between 1:1 and 1:6, or between 1:1 and 1:5, or between 1:1 and 1:4, or between 1:1 and 1:3, or between 1:1 and 1:2, or between 1:2 and 1:6, or between 1:3 and 1:5 grams of cell pellet to milliliter of preservation media. In some embodiments, the bacterial cell pellet is resuspended in a suitable aqueous preservation medium, where the weight ratio of cell pellet wet weight (grains) to preservation media (mL) can be 1:1, 1:2, 1:3, 1:4, 1:5, 1:6, 1:7, or 1:8 grams of cell pellet to milliliter of preservation media. In some embodiments, the bacterial cell pellet is resuspended in a suitable aqueous preservation medium, where the weight ratio of cell pellet wet weight (grams) to preservation media (mL) can be between 1:1 and 1:5 grams of cell pellet to milliliter of preservation media. In some embodiments, the bacterial cell pellet is resuspended in a suitable aqueous preservation medium, where the weight ratio of cell pellet wet weight (grams) to preservation media (mL) can be between 1:1 and 1:3 grams of cell pellet to milliliter of preservation media.

The aqueous preservation medium is comprised of ingredients that minimize the damaging effects encountered during the preservation process. The preservation medium of this invention includes a carbohydrate, a polyol, an anti-oxidant, a buffering agent, and, optionally, an amino acid. The carbohydrate used in the preservation medium functions as a lyoprotectant to protect and stabilize the cells during freeze drying, and afterwards during storage. Non-limiting exemplary carbohydrates suitable for use with the invention include trehalose, dextrose, lactose, maltose, sucrose and/or any other disaccharide or polysaccharide. In some embodiments, the preservation medium comprises from about 0.5% to about 30% carbohydrate by weight per volume (w/v) of the preservation medium, or from about 1% to about 25%, or from about 5% to about 20%, or from about 10% to about 15% carbohydrate by w/v of the preservation medium. In some embodiments, the preservation medium comprises from about 0.5% carbohydrate by weight per volume (w/v) of the preservation medium, or from about 1, 2, 5, 7, 10, 15, 20, 25, or 30% carbohydrate by w/v of the preservation medium. In some embodiments, the preservation medium comprises from about 5% to about 20% trehalose w/v of the preservation medium. In some other embodiments of the invention, the preservation medium comprises from about 5% to about 15% trehalose w/v of the preservation medium.

The polyol (i.e., polyhydric alcohol) of the preservation medium is a lyoprotectant that helps protect cells from the stresses of dehydration during freeze drying. Non-limiting exemplary polyols suitable for use with the present invention include xylitol, adonitol, glycerol, dulcitol, inositol, mannitol, maltitol, isomalt, lactitol, erythritol, sorbitol and/or arabitol. In some embodiments, the preservation medium comprises from about 0.1% to about 12% polyol by weight per volume (w/v) of the preservation medium, or from about 1% to about 10%, or from about 2% to about 9%, or from about 3% to about 7% polyol by w/v of the preservation medium. In some embodiments, the preservation medium comprises from about 0.1% polyol by weight per volume (w/v) of the preservation medium, or from about 0.5, 1, 2, 3, 5, 6, 7, 8, 9, 10, 11, or 12% polyol by w/v of the preservation medium. In some embodiments, the preservation medium comprises from about 2% to about 9% xylitol w/v of the preservation medium. In some other embodiments of the invention, the preservation medium comprises from about 2% to about 7% xylitol w/v of the preservation medium.

The antioxidant of the preservation medium retards oxidative damage to the microbial cells during the preservation and storage process. Non-limiting exemplary antioxidants suitable for use with the instant invention include sodium ascorbate, ascorbic acid, palmityl ascorbate, propyl gallate and vitamin E (α-tocopherol). In some embodiments, the preservation medium comprises from about 0.1% to about 5% antioxidant by weight per volume (w/v) of the preservation medium, or from about 0.5% to about 3.0%, or from about 1.0% to about 2.0% antioxidant by w/v of the preservation medium. In some embodiments, the preservation medium comprises from about 0.1% antioxidant by weight per volume (w/v) of the preservation medium, or from about 0.3, 0.5, 1.0, 1.5, 2.0, 2.5, 3.0, 3.5, 4.0, 4.5, or 5.0% antioxidant by w/v of the preservation medium. In some embodiments, the preservation medium comprises from about 0.5% to about 1.5% sodium ascorbate w/v of the preservation medium. In some other embodiments of the invention, the preservation medium comprises from about 0.5% to about 1.5% sodium ascorbate w/v of the preservation medium.

Buffering agents suitable for use in the preservation medium enhance the stability and recovery of the bacteria cells. A buffering agent suitable for use in the preservation medium is a physiological agent that does not exert any toxic effects on the bacteria, vaginal epithelial cells or a female patient using a pharmaceutical composition. Non-limiting exemplary buffering agents suitable for use with the instant invention include sodium phosphate, disodium phosphate, potassium phosphate, sodium bicarbonate, histidine, arginine and sodium citrate. In some embodiments, the buffering agent can have a pKa of from about 4.3 to about 8.0, or from about 4.6 to about 7.7, or from about 5.0 to about 7.3, or from about 5.4 to about 7.0, or from about 6.0 to about 6.7. In some other embodiments, the preservation medium comprises a buffering solution having a pKa of at least 4.3, 4.4, 4.5, 4.6, 4.7, 4.8, 4.9, 5.0, 5.5, 6.0, 6.5, 7.0, 7.5, 8.0, or higher. In some embodiments, the preservation medium comprises a buffering solution having a pKa in the physiological range. In other embodiments, the preservation medium comprises a buffering solution having a pKa of from about 6.7 to about 7.8.

In still further embodiments, the preservation medium comprises from about 5 mM to about 70 mM buffering agent, or from about 10 mM to about 65 mM, or from about 15 mM to about 60 mM, or from about 20 mM to about 55 mM, or from about 25 mM to about 50 mM, or from about 30 mM to about 45 mM, or from about 35 mM to about 40 mM buffering agent. In some embodiments, the preservation medium comprises from about 5 mM buffering agent, or from about 10, 15, 20, 25, 30, 35, 40, 45, 50, 55, 60, 65 or 70 mM. In some embodiments, the preservation medium comprises from about 10 mM to about 50 mM sodium phosphate. In some other embodiments of the invention, the preservation medium comprises horn about 10 mM to about 30 mM sodium phosphate.

In some embodiments, the preservation medium can optionally include an amino acid that helps enhance stability of the L. crispatus cells at elevated temperatures without significantly affecting cryopreservation during the lyophilization process. In some embodiments, the optional amino acid can be in the salt form of a suitable amino acid. Non-limiting exemplary amino acids and/or their salts suitable for use with the instant invention include sodium glutamate, glutamine, glycine, arginine, histidine, and lysine. In some embodiments, the preservation medium optionally comprises from about 0% to about 5% amino acid by weight per volume (w/v) of the preservation medium, or from about 0.5% to about 3.0%, of from about 1.0% to about 2.0% amino acid by w/v of the preservation medium. In some embodiments, the preservation medium optionally comprises from about 0.1% amino acid by weight per volume (w/v) of the preservation medium, or from about 0.3, 0.5, 1.0, 1.5, 2.0, 2.5, 3.0, 3.5, 4.0, 4.5, or 5.0% amino acid by w/v of the preservation medium. In some embodiments, the amino acid optionally included in the preservation medium is amino acid salt sodium glutamate, preferably monosodium glutamate. In some embodiments, the preservation medium optionally comprises from about 0% to about 5% sodium glutamate w/v of the preservation medium. In some other embodiments of the invention, the preservation medium optionally comprises from about 0% to about 5% monosodium glutamate w/v of the preservation medium. In some embodiments, the preservation medium optionally comprises from about 1% to about 4% sodium glutamate w/v of the preservation medium. In some other embodiments of the invention, the preservation medium optionally comprises from about 1% to about 4% monosodium glutamate w/v of the preservation medium.

The preservation medium of the present invention includes a carbohydrate that is between about 5% and 20% of the preservation medium by weight per volume, a polyol that is between about 2% and 9% of the preservation medium by weight per volume, an antioxidant that is between about 0.5% and 1.5% of the preservation medium by weight per volume and a buffering agent that is between 10 mM and 50 mM. In other embodiments, a preservation medium suitable for use with the present invention can include a carbohydrate that is between about 5% and 15% of the preservation medium by weight per volume, a polyol that is between about 2% and 7% of the preservation medium by weight per volume, an antioxidant that is between about 0.5% and 1.0% of the preservation medium by weight per volume and a buffering agent that is between 10 mM and 30 mM.

In some embodiments, the preservation medium of the present invention includes a carbohydrate that is between about 5% and 20% of the preservation medium by weight per volume, a polyol that is between about 2% and 9% of the preservation medium by weight per volume, an antioxidant that is between about 0.5% and 1.5% of the preservation medium by weight per volume, a buffering agent that is between 10 mM and 50 mM, and, optionally, an amino acid that is between about 0% and 5% of the preservation medium by weight per volume. In other embodiments, a preservation medium suitable for use with the present invention can include a carbohydrate that is between about 5% and 15% of the preservation medium by weight per volume, a polyol that is between about 2% and 7% of the preservation medium by weight per volume, an antioxidant that is between about 0.5% and 1.0% of the preservation medium by weight per volume, a buffering agent that is between 10 mM and 30 mM and, optionally, an amino acid that is between about 0% and 5% of the preservation medium by weight per volume.

An example of a particularly useful preservation medium of the present invention includes trehalose as the carbohydrate that is between about 5% and 20% of the preservation medium by weight per volume, xylitol as the polyol that is between about 2% and 9% of the preservation medium by weight per volume, sodium ascorbate as the antioxidant that is between about 0.5% and 1.5% of the preservation medium by weight per volume and sodium phosphate as the buffering agent that is between 10 mM and 50 mM. In some embodiments, a particularly useful preservation medium of the present invention includes trehalose as the carbohydrate that is between about 5% and 20% of the preservation medium by weight per volume, xylitol as the polyol that is between about 2% and 9% of the preservation medium by weight per volume, sodium ascorbate as the antioxidant that is between about 0.5% and 1.5% of the preservation medium by weight per volume, sodium phosphate as the buffering agent that is between 10 mM and 50 mM and, optionally, sodium glutamate as the amino acid that is between about 0% and 5% of the preservation medium by weight per volume. In other embodiments, a preservation medium suitable for use with the present invention includes trehalose that is between about 5% and 15% of the preservation medium by weight per volume, xylitol that is between about 2% and 7% of the preservation medium by weight per volume, sodium ascorbate that is between about 0.5% and 1.0% of the preservation medium by weight per volume and sodium phosphate that is between 10 mM and 30 mM. In some other embodiments, a preservation medium suitable for use with the present invention includes trehalose that is between about 5% and 15% of the preservation medium by weight per volume, xylitol that is between about 2% and 7% of the preservation medium by weight per volume, sodium ascorbate that is between about 0.5% and 1.0% of the preservation medium by weight per volume, sodium phosphate that is between 10 mM and 30 mM, and, optionally, sodium glutamate that is between about 0% and 5% of the preservation medium by weight per volume. Representative preservation media compositions, which are in no way meant to be limiting, are included in Table 1 below:

TABLE 1
Exemplary preservation media compositions and ingredient ratios

| | Ingredient (%. w/w) | | | | |
|---|---|---|---|---|---|
| | | | Sodium | | Sodium |
| No. | Trehalose | Xylitol | ascorbate | NaPO₄ * | glutamate |
| i | 5-15 | 2-9 | 0.5-1.5 | 10-30 | 0-5 |
| ii | 5, 10, | 2 | 0.5 | 10 | 0-5 |
| | or 15 | | | | |
| iii | 7.5 | 2, 3, | 0.75 | 15 | 0-5 |
| | | or 9 | | | |
| iv | 7.5 | 3 | 0.5, 1.0, | 15 | 0-5 |
| | | | or 1.5 | | |
| v | 7.5 | 3 | 0.75 | 10, 12, | 0-5 |
| | | | | or 15 | |

* Amount of sodium phosphate is measured in mM

Prior to addition of the above described harvested L. crispatus cells to the medium, the cells may be washed in a phosphate-buffered saline solution. Upon introduction of the harvested L. crispatus cells to the preservation medium described herein, the resulting mixture is referred to as the cell-preservation medium slurry. In some embodiments, a cell-preservation medium slurry can have an activity of between 10⁸ CFU/mL and 10¹¹ CFU/mL. A more preferred cell-preservation medium slurry can have an activity of at least about 10¹⁰ CFU/mL. It is to be understood that one of ordinary skill in the art will appreciate variations to the basic culturing, harvesting and suspending steps disclosed herein and as such, the present invention incorporates such variations.

### Drying the Cell-Preservation Medium Slurry

The cell-preservation medium slurry can be dried to produce the resulting bulk drug powder using any suitable drying method known in the art. Typically the effect of drying is to place the bacteria in a state of dormancy to protect the bacteria from environmental elements that negatively impact the viability of the bacteria. The standard way to bring about dormancy is through the removal of water. Generally, sufficient water is removed so that the normal cellular processes (e.g. enzymatic activity) come to a halt or are at least greatly diminished.

The cell-preservation medium slurry can be dried using any of the numerous methods known in the art for drying a bacterial preparation to increase their stability for long term storage. Drying methodologies and protective agents are disclosed in the review by Morgan et al. (2006) J. Microbiol. Meth. 66:183-193. Suitable drying methods include air drying, vacuum drying, oven drying, spray drying, flash drying, fluid bed drying, controlled atmosphere drying, and lyophilization (i.e., freeze drying). In some embodiments, a desiccant is used to aid in the drying process, and/or to prevent reabsorption of moisture into the dried formulation. In some embodiments, the drying is carried out using a lyophilizer (i.e., Virtis, SP Scientific). Detailed freeze-drying methods known to persons of skill in the art and are disclosed in U.S. Pat. Nos. 6,093,394; 8,329,447; and 8,642,029. The resulting dry formulation referred to as the bulk powder is tested for potency using the methods described below. The potency of the dry bulk drug powder can be between 10⁹ CFU/g and 10¹² CFU/g. A more preferred bulk powder can have an activity of at least about 10¹⁰ CFU/g.

### Measuring Residual Water

A dried formulation can be tested for the presence of residual water using any suitable method known in the art. In some cases, residual water in the dried formulation can be measured gravimetrically, as described in U.S. Pat. Nos. 8,329,447 and 8,642,029. Alternatively, an instrument for measuring water content in powders could be used to monitor the moisture content of the formulation during drying, e.g., the IR-120 Moisture Analyzer (Denver Instruments, Denver, Colo.). Residual water moisture can also be determined by performing well known coulometric or volumetric titration techniques, such as the Karl Fischer titration.

Water content in a Lactobacillus powder can also be measured as the free water or water activity (a_{w}) using a water activity meter, e.g., AquaLab CX-2 Model series (Decagon Instruments, Pullman, Wash.), or a Rotronic Model series (Rotronic Instillment Corp., Huntington, N.Y.). The water activity meter (AquaLab CX-2, Decagon Instruments) uses a chilled-mirror dew point technique to measure the a_{w} of a product. When a sample is placed in the AquaLab, a stainless-steel mirror within the chamber is repeatedly cooled and heated while dew forms and is driven off. The instrument's fan circulates the air in the sensing chamber, speeding up the equilibration process. Each time dew forms on the mirror, AquaLab measures the temperature and calculates the a_{w} of the sample, saving these values to compare to previous values. When the a_{w} values of consecutive readings are less than 0.001 apart, the measurement process is complete.

The water energy level or water activity (a_{w}) contributes to the overall stability of the resulting dry bulk Lactobacillus drug powder. One of ordinary skill in the art will appreciate the importance of the water activity of pharmaceuticals, such as the a_{w} of the drug powder of the invention. By maintaining a low water activity of a pharmaceutical product, degradation of the active pharmaceutical ingredient (i.e., the L. crispatus drug powder) can be avoided. Furthermore, a pharmaceutical product, such as the Lactobacillus drug powder of the present invention, having a low water activity can be less susceptible to crystallization, caking and clumping, which contributes to the drug's degradation and ineffectiveness. These are time-dependent reactions with rates influenced by water activity. Details on the influence of a_{w} on a product formulation can be found in United States Pharmacopeial Method <1112> Microbiological Attributes of Non-sterile Pharmaceutical Products—Application of Water Activity Determination.

In some embodiments, the dry bulk L. crispatus drug substance can have a measured a_{w} of from about 0.001 to about 0.220, or from about 0.005 to about 0.200, or from about 0.010 to about 0.150, or from about 0.025 to about 0.100, or from about 0.050 to about 0.075. In other embodiments, the dry bulk Lactobacillus drug substance can have a measured a_{w} of from about 0.001, 0.003, 0.005, 0.007, 0.010, 0.030, 0.050, 0.070, 0.100, 0.150, 0.170, 0.200, 0.220. In particular embodiments, the dry bulk L. crispatus drug substance can have a measured a_{w} of less than 0.220.

### Measuring Potency

The L. crispatus formulations of the present invention are tested for potency at different times throughout the preparation process using any suitable method known in the art. Such methods used to determine the potency that of the L. crispatus formulations include, but are not limited to, the culture-based method. The light scattering method for determining cell density of L. crispatus is used to monitor the fermentation process and involves measuring the optical density at 600 nm of a sample of bacteria.

The preferred method used to measure the potency of the L. crispatus formulations is the culture-based method involving serial dilutions. A sample of the L. crispatus formulation to be tested is obtained and serial dilutions are made. A small aliquot (i.e., 100 μL) of serial dilutions is plated onto MRS agar plates. The samples are allowed to incubate anaerobically at 37° C. for 48 hours. After a suitable amount of time has passed, the plates are illuminated by placing the Petri dishes in transmitted light. The separate colonies are counted manually or with a camera and computer using commercially available bacterial counting software, and the number of CFUs in the samples are calculated as CFU/ml or CFU/gram. More details involving the culture-based methods are disclosed in Brugger, S. D., et al. Automated Counting of Bacterial Colony Forming Units on Agar Plates. PLOS ONE 2012; 7 (3): e33695.

### Purity and Identity

In addition to measuring the potency, the composition/powder herein disclosed can be tested for purity and identity. The purity is determined using methods well known in the art and as described in United States Pharmacopeial Method <61> Microbial Enumeration Tests and United States Pharmacopeial Method <62> Tests for Specified Microorganisms. Genetic identification of the L. crispatus species in the composition/powder herein disclosed is carried out by isolating genomic DNA using a commercially available kit (e.g. PowerSoil DNA Isolation Kit, Qiagen), amplifying the 16S ribosomal RNA gene using specific primers by PCR, sequencing the gene using a commercial DNA sequencing service (MCLAB), and comparing the sequence to a reference standard. Identification of the L. crispatus strain in the composition/powder herein discloses is determined using methods well known in the art, such as Repetitive Sequence Polymerase Chain Reaction (Rep PCR) and as described in U.S. Pat. Nos. 60,933,941; 8,329,447; and 8,642,029.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The following examples are provided by way of illustration only and not by way of limitation. Those of skill will readily recognize a variety of noncritical parameters which could be changed or modified to yield essentially similar results.

### EXAMPLES

### Example 1—Clinical Protocol for a Pregnant Subject Receiving the Composition Herein Disclosed

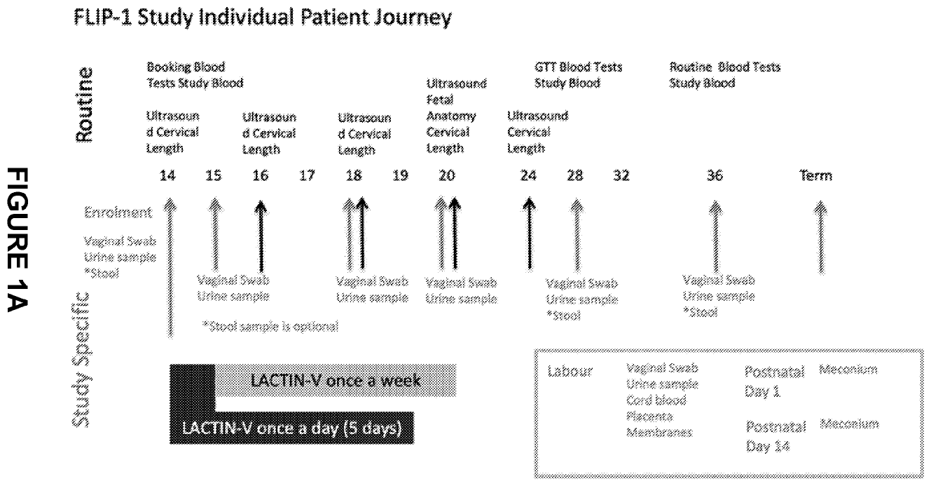

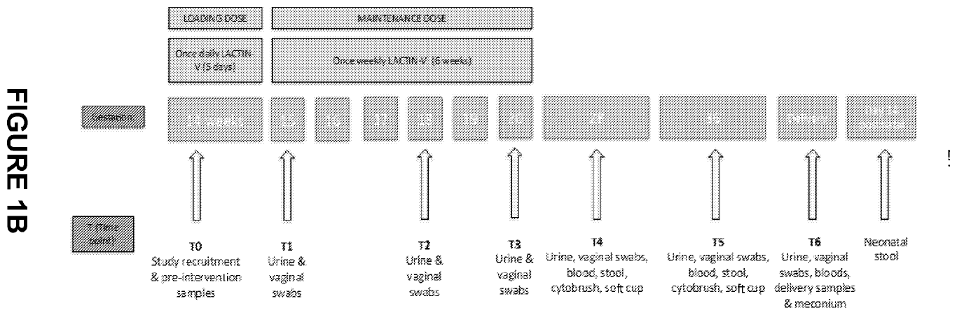

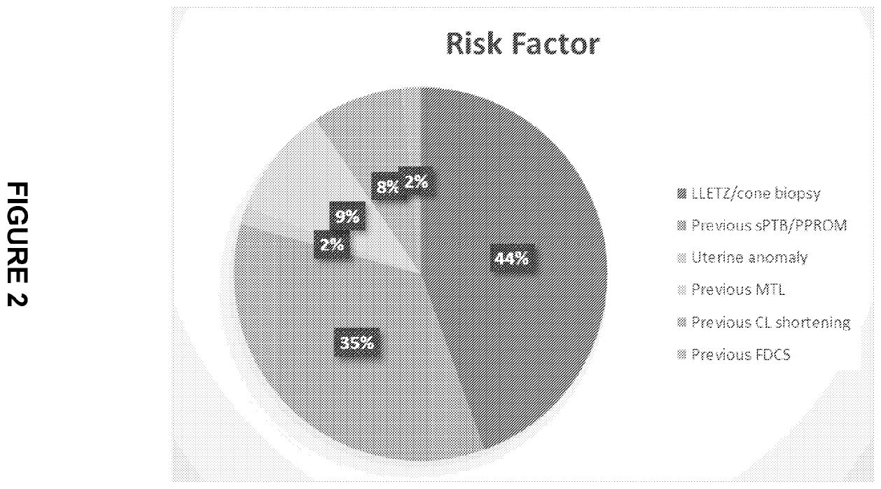

The composition for use herein disclosed may be used in the clinical protocol described below and in FIGS. 1A and 1B.

Following enrolment at or as close to 14 weeks as possible, the subject will be given a “loading phase” of 5 daily doses of L. crispatus CTV-05 (LACTIN-V), followed by a “maintenance phase” of 6 weekly doses, for a total of 11 doses. The subject will be administered the composition via a self-administered vaginal applicator.

### Example 2—Preparation of the Composition of L. Crispatus

This example details the general strategy for preparing the dry composition of L. crispatus cells in powder form, involving bacterial cultivation, suspension in preservation medium, drying, dilution, and packaging. The procedure described here, for the culture and processing of L. crispatus SJ-3C, is applicable for any microorganism suitable for use with the present invention, for example L. crispatus CTV-05.

The initial L. crispatus SJ-3C (SJ-3C) cells can be obtained from the deposit under ATCC number PTA-10138. The L. crispatus CTV-05 cells can be obtained from the deposit under ATCC accession number 202225. The L. crispatus strains MV-3A-US and/or MV-1A-US can be obtained through BEI Resources (NIH/NIAID). A Master Cell Bank and Working Cell Bank of these cells are prepared and can be subsequently used in the preparation of the dry L. crispatus compositions.

The SJ-3C cells are initially plated onto modified MRS agar plates and grown under anaerobic conditions for 72 hours at 37° C. Cells from the plates are inoculated into 10 mL of modified MRS and incubated anaerobically for 24 hours at 37° C. This culture is then transferred to 490 mL of growth medium and incubated for 24 hours at 37° C., followed by transfer to 4.5 L of medium in a 5 L Bellco Bioreactor. The 5-liter culture is incubated anaerobically at 37° C. for an additional 24 hours to serve as the fermentor inoculum.

Fermentation is performed in a fermentor (100 L fermentor) at pH 6.0 in the presence of modified MRS medium sparged with nitrogen gas. Fermentation is initiated by addition of the inoculum and completed after approximately 15 hours when the cells reach early stationary phase and growth stops. At this point, glucose is depleted, lactic acid production stops, the optical density of the culture at 600 nm (OD600) remains constant and the cells are harvested.

Cells are harvested, concentrated, and washed by buffer exchange into phosphate-buffered saline (diafiltration) in a sterile closed hollow fiber system using a tangential flow membrane. When the residual lactate concentration reaches 10% of the starting value at harvest and pH of the permeate remains constant, the cells are aseptically removed from the harvest system and collected by centrifugation at 1500×g for 20 minutes, 2-8° C.

Cell pellets are resuspended in a preservation medium solution, using 2.5 mL of preservation solution per gram of cell paste. The preservation medium solution contains 15% trehalose, 6% xylitol, and 1% sodium ascorbate in a 10 mM sodium phosphate buffer (pH 7.4), which is used to prepare batches of the harvested SJ-3C slurry. The resulting batches of the preservation medium cell slurry are to have calculated activities of between 1×10¹⁰ CFU/mL and 5×10¹⁰ CFU/mL. The slurry is transferred to sterile Lyoguard™ trays and lyophilized in a Virtis Genesis Lyophilizer. Viability of the cell slurry is determined prior to lyophilization by plate counting. The Lyoguard™ trays containing the cell cakes are placed in heat-sealed bags with desiccant and purged with nitrogen gas and held at 2-8° C. until milling.

The SJ-3C bulk drug substance is produced by milling the lyophilized cell cakes with 0.5% colloidal silicon dioxide as an anti-caking agent using a cone mill. The bulk powder is purged with nitrogen (N₂) gas and stored with desiccant in a heat-sealed bag at 2-8° C. until used for manufacture of the drug product. The SJ-3C bulk drug substance is tested for purity, potency (CFU), identity, and residual moisture using the methods as described previously and those known to one of skill in the art. The ideal activity of the resultant batches of the dry SJ-3C bulk drug substance should be between 5×10¹⁰ CFU/g and 1.0×10¹¹ CFU/g. The ideal water activity of the dry SJ-3C bulk drug substance should be <0.220. When tested for purity, the resulting SJ-3C bulk drug substance will contain <200 CFU/g of total aerobic counts, <20 CFU/g of total yeasts and molds, and an absence of objectionable organisms. The identity of the resulting SJ-3C bulk drug substance is confirmed by the 16S ribosomal RNA gene sequence.

The bulk drug substance may be diluted by 3 to 10-fold with maltodextrin to give a final dose of 2×10⁹ CFU/dose to 5×10⁹ CFU/dose. The dose is 200 mg. One dose of the diluted drug substance may be placed in a medical powder applicator and packaged as the final drug product.

### Example 3—Clinical Outcomes of Pregnant Women Receiving LACTIN-V

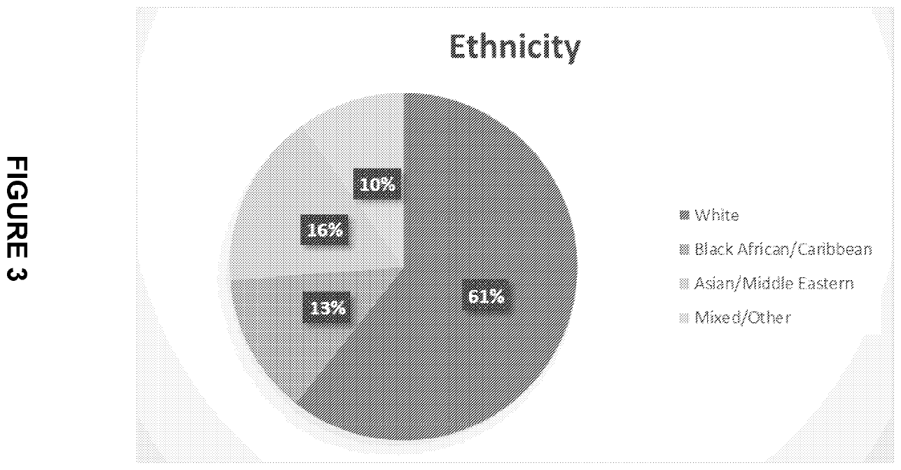

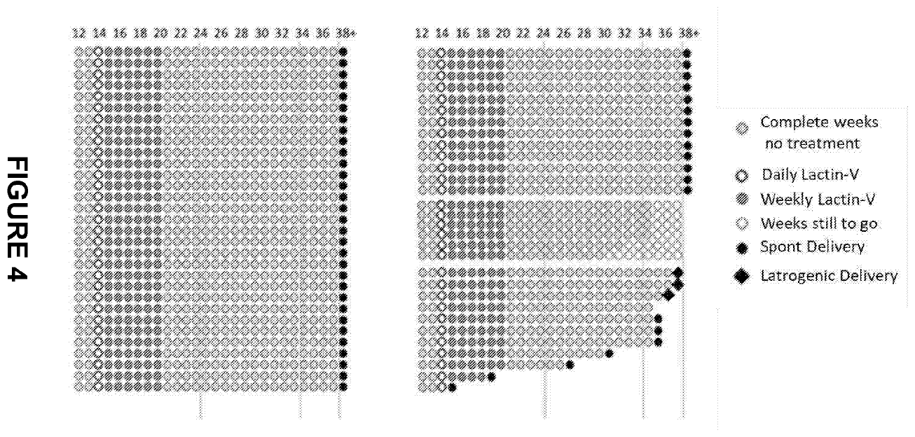

Out of 61 total active participants undergoing the above described clinical protocol, 61 women have either given birth or miscarried. The proportion of the risk factors and ethnicity factors for the 61 total active participants are shown in FIGS. 2 and 3.

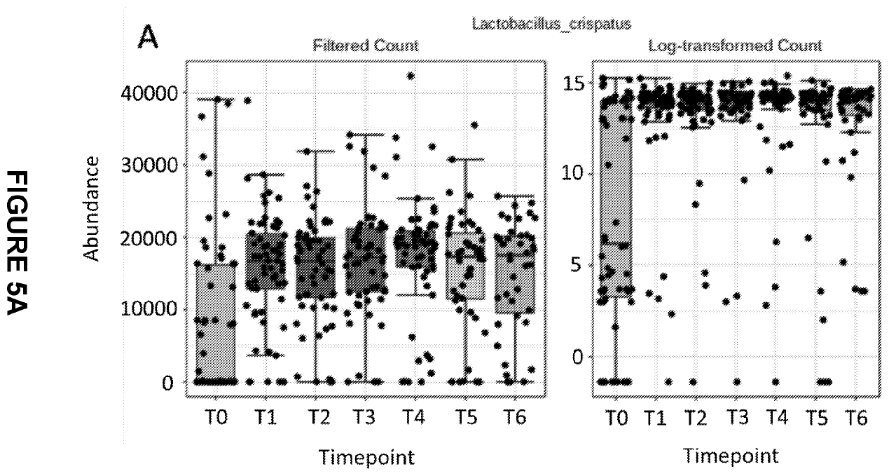

Out of the 61 women, 48 of them carried their babies to full term, 2 women miscarried, 6 women had spontaneous PTB and 5 women had iatrogenic PTB (FIG. 4). No unsolicited adverse events or serious adverse events were reported, demonstrating the safety and tolerability of LACTIN-V in pregnant subjects.

The rate of spontaneous PTB in the LACTIN-V treated cohort <34 weeks is 3.3% and <37 weeks is 9.8%. This compares to PTB rate <34 weeks of 7.0% and <37 weeks of 17.8% from data collected from 2002 to 2020 in a similar population who were not receiving LACTIN-V.

Accordingly, as is demonstrated from the exemplified data herein disclosed, the present invention surprisingly teaches that the use of a high potency L. crispatus strain in a high viability formulation can yield clinically significant results, i.e. a reduction in PTB rates, without the need for concomitant antibiotic therapy.

### Example 4—Microbiological Outcomes of Pregnant Women Receiving LACTIN-V

PCR primers were specifically developed for identifying LACTIN-Vin the vaginal samples of the enrolled pregnant women. In a preliminary study to determine their effectiveness, LACTIN-Vwas detected in 6 out of 7 samples taken from patients during the weekly treatment phase of the study and in 4 of 8 samples taken later in the third trimester after administration of LACTIN-Vhad discontinued. This demonstrates persistence of LACTIN-Vfor at least a week in the majority of cases, and for several weeks following last administration in at least 50% of cases.

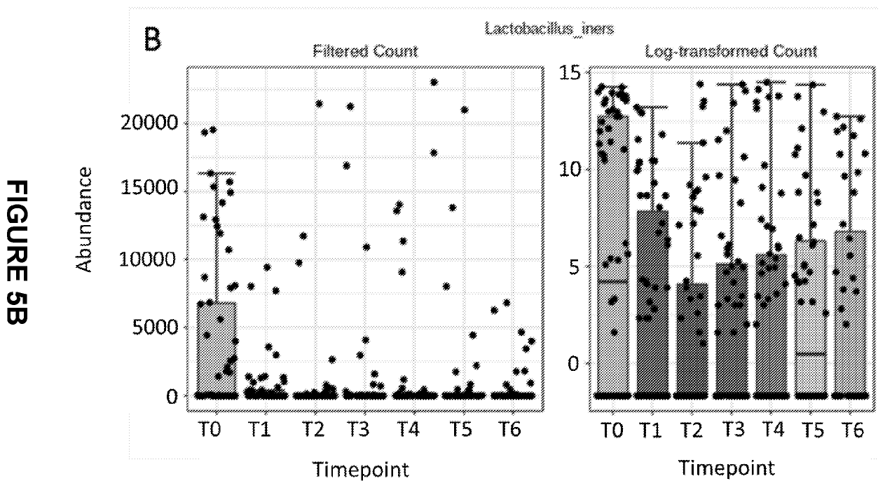

Metataxonomics analysis of vaginal microbiota showed that the relative abundance of Lactobacillus crispatus increased markedly following administration of LACTIN-V while the abundance of Lactobacillus iners decreased (FIG. 5 in combination with FIG. 1B). This effect was sustained until delivery, well after the last dose of LACTIN-V. This equated to a shift in the prevalence of L. crispatus-dominated vaginal microbiota from 35% pre-LACTIN-V therapy, to 95% post-loading dose. Since Lactobacillus iners has been associated with cervical shortening and PTB, these results suggest a potential mechanism whereby LACTIN-V can prevent PTB by promoting L. crispatus dominance of the vaginal microbiota.

Of the 61 patients enrolled, 10 had microbiological evidence of bacterial vaginosis at some point during the study, 8 of these had resolved with LACTIN-V therapy.

Of 12 patients who had detectable group B Streptococcus (on culture) at some point in pregnancy, 7 demonstrated resolution by 36 weeks and only 1 had group B Streptococcus detected on delivery vaginal swabs.

The invention claimed is:

1. A method increasing the probability of full term birth in a pregnant female subject,
comprising administering to the subject a composition comprising Lactobacillus crispatus cells by topical vaginal application,
wherein the L. crispatus cells functionally express a pullulanase gene to utilize glycogen and produce both L- and D-lactic acid, and
wherein the subject has not been pretreated with an antibiotic.

2. The method of claim 1, wherein the L. crispatus cells produce hydrogen peroxide.

3. The method of claim 1, wherein the composition comprises the L. crispatus cells as a lyophilised powder.

4. The method of claim 1, wherein the composition produces an amount of viable cells between 1×10⁸ and 1×10¹⁰ CFU per dose when plated on MRS agar.

5. The method of claim 1, wherein the subject is planning a pregnancy or is in the first or second trimester of pregnancy.

6. The method of claim 1, wherein the subject has a prior history of preterm birth; and/or a short cervix of 25 mm or less in length.

7. The method of claim 1, wherein a vaginal fluid sample obtained from the subject has a total bacterial population, and at least 50% of the total bacterial population is determined to comprise Lactobacillus iners.

8. The method of claim 1, wherein a vaginal fluid sample obtained from the subject has a total bacterial population, and less than 50% of the total bacterial population comprises Lactobacillus spp.

9. The method of claim 1, wherein the L. crispatus is L. crispatus strain CTV-05 deposited under ATCC accession number 202225.

10. The method of claim 1, wherein the L. crispatus is L. crispatus strain SJ-3C deposited under ATCC accession number PTA-10138.

11. The method of claim 1, wherein the L. crispatus is L. crispatus strain MV-3A-US and/or MV-1A-US.

12. The method of claim 1, wherein the composition is administered to a subject who has previously been administered a powder comprising Lactobacillus crispatus cells.

13. The method of claim 1, wherein the composition is administered to the subject up to 34 weeks after last menstrual period (LMP).

14. The method of claim 1, wherein the composition is administered daily for 2 to 7 days and then once or twice weekly thereafter.

15. The method of claim 1, wherein the antibiotic is an antibiotic targeting vaginal bacteria.

16. The method of claim 4, wherein the composition produces an amount of viable cells between 5×10⁸ and 5×10⁹ CFU per dose when plated on MRS agar.

17. The method of claim 5, wherein the subject is early in the first trimester of pregnancy.


SEQUENCE LISTING (4 sequences)

SEQ ID NO: 1 - protein, 1251 aa, Lactobacillus crispatus
  Strain SJ-3c
MNKKSGHNIKFKSIFVCTSAIMSLWLGANLTTTQVHAAEDNAAPKSSEVVGQTNSSKDNAATATVQNQSNAKAKQRQQGVAPQNVPTVLAAKTKDEGTNNSPAREEVKDQTKVVIHYNGDGEKWVPYIWGKKPNGNGNEYKWAGKDDNGYYSVIDLDKNYDQVGVLIKTKGSWAGKDGTGADRSLNVSDNGKAEVFYKAGSDDAQKVTPEYHSANINLHYYGDDQVNKVNVWTDKDQDNKQLISLTKQGNSQDGSIKLDNTDFSKLFVAPVGSDEITREFTPLPGNKATDIYLVKNDTEAYYSKSFALADQSLSSAYMTSPDSLTVEAGKATTVDKIKGKLSLRNNSIDHIEAVDANADGKAKKFIIHTKTDIDILKDNQVGLGMNFKNVDIGPYVRSKAFDDKYAYDGDDLGCTYDSKKTQIKLWAPTAKDVELNLYDSVDNSAKPTNVIALTRGDRGVWTGIIKGDKKGWAYDFKLTFGNGKITQTDDPYSKAVTVNGVRSVIEDYNNIKPTDFNRMPSFSKPTDAIVYEISIRDFSSDPNSGIKDKGKFLGMIESGKTPTGQVTGLDYLKALGITHVQIMPSYDFASIDETKSLKNQYNWGYDPKNYDVPEGSYSSDPTNPTARIIEMKEMINGLHKAGIRVIMDVVYNHVYNPQEQAFELTVPGYYFRYDADNNTTSDSGCGNDIGSQRKMVRKYIVDSVVYWAKNYNLDGFRFDIMSLLDRETMNDVRAALDKIDPGIITYGEGWEVGNQKYTREEGTTQYTADKVPNVGFFSDDMRNSVKGNNDGSEPGLVIGNGNEKNFDKHAVLFTDSFLRGKWLHNDSWEPHNYATPSQMVNYAACHDGKTLYDYIKAAMPNESDENIARRVRLANSMIMLSQGISFFHSGQEALRTKGGNSNSYNSPVNINEINWERVGANKASVQYFQKLAKLRKQLPILHLNDFNKIYDENVTKIISYGRETKDKNIKGVFEYEYNVNGKKLLIVFNVNNDKVALNNVDLSHGKKLLDSDGNVKLSDKTMLAPLSTLVVDEDGTMVLPDVPKPDQPVSPTTPGDKPGKPTTPENPRIPDQKPANPIVPQETIITPEAPSENSIIDNTKNIINGSEETTTVSTQDNTTTEILLTHNAYVYDSEANPIYQNDNKLVLKAGDTIKPLDNGKKYSIKGKLFYKVAKNQFVKVANTVSYYKLAHNSFVYNKHGKAVKKNGKRILLKKNKHISLHSDKVVKIKGKKFYQLKDGSFIKARNVKRIG

SEQ ID NO: 2 - protein, 1259 aa, Lactobacillus crispatus
  Strain CTV-05
MILWRNLFMNKKSGHNIKFKSIFVCTSAIMSLWLGANLTTTQVHAAEDNAAPKSSEVVGQTNSSKDNAATATVQNQSNAKAKQRQQGVAPQNVPTVLAAKTKDEGTNNSPAREEVKDQTKVVIHYNGDGEKWVPYIWGKKPNGNGNEYKWAGKDDNGYYSVIDLDKNYDQVGVLIKTKGSWAGKDGTGADRSLNVSDNGKAEVFYKAGSDDAQKVTPEYHSANINLHYYGDDQVNKVNVWTDKDQDNKQLISLTKQGNSQDGSIKLDNTDFSKLFVAPVGSDEITREFTPLPGNKATDIYLVKNDTEAYYSKSFALADQSLSSAYMTSPDSLTVEAGKATTVDKIKGKLSLRNNSIDHIEAVDANADGKAKKFIIHTKTDIDILKDNQVGLGMNFKNVDIGPYVRSKAFDDKYAYDGDDLGCTYDSKKTQIKLWAPTAKDVELNLYDSVDNSAKPTNVIALTRGDRGVWTGIIKGDKKGWAYDFKLTFGNGKITQTDDPYSKAVTVNGVRSVIEDYNNIKPTDFNRMPSFSKPTDAIVYEISIRDFSSDPNSGIKDKGKFLGMIESGKTPTGQVTGLDYLKALGITHVQIMPSYDFASIDETKSLKNQYNWGYDPKNYDVPEGSYSSDPTNPTARIIEMKEMINGLHKAGIRVIMDVVYNHVYNPQEQAFELTVPGYYFRYDADNNTTSDSGCGNDIGSQRKMVRKYIVDSVVYWAKNYNLDGFRFDIMSLLDRETMNDVRAALDKIDPGIITYGEGWEVGNQKYTREEGTTQYTADKVPNVGFFSDDMRNSVKGNNDGSEPGLVIGNGNEKNFDKHAVLFTDSFLRGKWLHNDSWEPHNYATPSQMVNYAACHDGKTLYDYIKAAMPNESDENIARRVRLANSMIMLSQGISFFHSGQEALRTKGGNSNSYNSPVNINEINWERVGANKASVQYFQKLAKLRKQLPILHLNDFNKIYDENVTKIISYGRETKDKNIKGVFEYEYNVNGKKLLIVFNVNNDKVALNNVDLSHGKKLLDSDGNVKLSDKTMLAPLSTLVVDEDGTMVLPDVPKPDQPVSPTTPGDKPGKPTTPENPRIPDQKPANPIVPQETIITPEAPSENSIIDNTKNIINGSEETTTVSTQDNTTTEILLTHNAYVYDSEANPIYQNDNKLVLKAGDTIKPLDNGKKYSIKGKLFYKVAKNQFVKVANTVSYYKLAHNSFVYNKHGKAVKKNGKRILLKKNKHISLHSDKVVKIKGKKFYQLKDGSFIKARNVKRIG

SEQ ID NO: 3 - DNA, 20 bp, Artificial Sequence
  Description of sequence Primer AM51
cggtccttatgttcgttcga

SEQ ID NO: 4 - DNA, 20 bp, Artificial Sequence
  Description of sequence Primer AM52
cacctggagtggttggactt